(19) United States
(12) Patent Application Publication
(10) Pub. No.: US 2025/0215351 A1
(43) Pub. Date: Jul. 3, 2025

(54) COMPOSITION AND METHOD

(71) Applicant: INNOSPEC ACTIVE CHEMICALS LLC, High Point, NC (US)

(72) Inventors: Mitchell Jared COHEN, Winston Salem, NC (US); Stephen Moss O'CONNOR, Davidson, NC (US); Michael Cameron HOWARD, High Point, NC (US)

(21) Appl. No.: 19/002,096

(22) Filed: Dec. 26, 2024

Related U.S. Application Data
(60) Provisional application No. 63/615,041, filed on Dec. 27, 2023.

(51) Int. Cl. C11D 1/14; C11D 3/20; C11D 17/00

(52) U.S. Cl. CPC C11D 1/143; C11D 3/2079; C11D 17/0047; C11D 2111/12; C11D 2111/14

(57) ABSTRACT

A solid cleansing composition including
(a) at least 35 wt % of one or more taurate surfactants of formula (I):

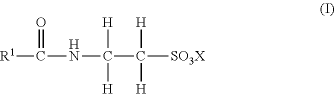

in which X is hydrogen, a metal ion or an optionally substituted ammonium ion; and R¹ is an optionally substituted C₃-C₃₅ hydrocarbyl group; and
(b) at least 15 wt % of one or more fatty acids.

The solid cleansing composition may be particularly useful as a personal care bar. A method of forming the solid cleansing composition and uses of the solid cleansing composition are also disclosed.

### FIELD

The present invention relates to a solid cleansing composition, suitably in bar form, to a method of forming such a solid cleansing composition and to uses of said solid cleansing composition. In particular the present invention relates to solid cleansing compositions comprising relatively high amounts of acyl taurate surfactants and fatty acids.

### BACKGROUND

Conventional soap bars comprise a large portion, typically 60-80% by weight of fatty acid soaps, often a mixture of fatty acid soaps, selected to obtain the required properties of lather, bar structure and wear properties. Such fatty acid soaps are typically alkali metal salts, especially sodium salts of fatty acids, having a chain length of 12 to 22 carbons atoms. Such conventional soap bars are manufactured by refining/milling, plodding and stamping a semi solid mass of soap and other components. This has become an efficient and relatively low-cost manufacturing process due to the equipment required to run the process being relatively simple and implemented at large scale around the world. Conventional soap bars may also be manufactured by solidifying a molten cleansing composition in a mould in a so-called “melt and pour” process.

Although cheap, effective and easy to manufacture, fatty acid soap can be drying to the skin and consumers are increasingly seeking milder alternatives. Therefore, manufacturers have tried to develop cleansing bars comprising alternative surfactants which are milder than fatty acid soaps. These alternative surfactants are generically known as “synthetic detergents” and cleansing bars comprising such alternative, mild surfactants are known as “syndet bars” or as “combi bars” when the bar also contains a significant amount of conventional fatty acid soap. One disadvantage of syndet and combi bars is that it has proved difficult to produce such bars using conventional soap making processes which would be efficient and low cost, and could be performed using equipment already in place at manufacturing sites. The processes by which syndet and combi bars often have to be produced are relatively high cost due to the need for expensive machinery and/or high energy inputs.

One class of such relatively mild surfactants for use in syndet and combi bars are the acyl isethionates, including sodium cocoyl isethionate (SCI). These acyl isethionates have been shown to be mild to the skin, to lather well and to produce good foam. Sodium acyl isethionates are widely used in syndet bar products and provide acceptable hardness, solubility and cleansing required of a cleansing bar.

However, acyl isethionates are considerably more expensive than fatty acid soap and as discussed above, syndet bars comprising acyl isethionates have proved difficult to produce in an efficient manner in conventional fatty acid soap bar manufacturing processes. It is therefore common practice to provide a cleansing bar which combines conventional fatty acid soap compounds with milder synthetic detergents such as isethionates. Such cleansing bars provide the desired properties of mildness and improved lather due to the presence of the synthetic surfactant but the cost is reduced by the inclusion of the fatty acid soap compounds.

Also, there is a need to improve the sustainability of cleansing compositions, driven both by consumer preference and the need to reduce the environmental impact of the manufacture of personal care products. Therefore, it is becoming increasingly important for manufacturers to assess the extent to which their products and ingredients used to make said products are obtainable from renewable sources. One way of measuring the sustainability of chemical products is to calculate percentage renewable carbon index (% RCI). This is done by dividing the number of carbon atoms in a chemical product derived from renewable carbon sources by the total number of carbon atoms in the product.

Commercially available sodium acyl isethionate surfactants have a % RCI of only up to 86%. Therefore, it is not possible to produce a cleansing composition solely from renewable sources when using current commercially available sodium acyl isethionate surfactants in the composition. Alternative mild surfactants with higher % RCI are therefore sought for sustainably manufacturing syndet bars.

Sodium acyl alkyl taurate surfactants, such as sodium methyl cocoyl taurate, have also been used in the manufacture of syndet bars. However, such acyl alkyl taurates are often only available as liquids or pastes, as manufactured, and are therefore difficult to formulate into a syndet bar having a low water content. Such acyl alkyl taurates are therefore often used as secondary surfactants in relatively small amounts in syndet bars. Such bars comprising acyl alkyl taurates often require the use of significant proportions of bar structurants, such as polyalkylene oxides. Also, such acyl alkyl taurates do not have a 100% or near 100% % RCI and therefore are not obtained from completely renewable chemical feedstocks.

Other classes of relatively mild surfactants are alkyl ether sulfates and glutamates. These surfactants have higher water solubility than fatty acid soaps and other synthetic surfactants and therefore also need to be combined with other surfactants in order to provide a cleansing bar which does not wear too quickly in use.

As such, there remains a need for a high performance syndet bar which can be efficiently manufactured, preferably using conventional soap making equipment and processes, from completely sustainable chemical feedstocks.

### SUMMARY OF THE INVENTION

It is one aim of the present invention to provide a solid cleansing composition which has Desirable properties, such as hardness, foaming and wear, expected of a personal cleansing bar, without using significant quantities of fatty acid soap components. Specifically, the present invention may aim to allow the incorporation of relatively high amounts of acyl taurate surfactants, to provide mild surfactant properties, whilst retaining the required physical properties of a solid bar cleansing composition. Taurate surfactants, such as acyl alkyl taurate surfactants, are often available only in liquid or paste form which is difficult to process into a solid cleansing composition, for example in bar form, without high amounts of structurants.

Furthermore, it is an aim of the present invention to provide such a solid cleansing composition which can be made using conventional soap making techniques and equipment, which may provide cost and efficiency advantages compared to current processes for manufacturing syndet bars.

It may be a further aim of the present invention to provide a solid cleansing composition which can provide a syndet cleansing bar having a relatively high percentage renewable carbon index, compared to known syndet cleansing bars.

According to aspects of the present invention, there is provided a solid cleansing composition, a method and use as set forth in the appended claims. Other features of the invention will be apparent from the dependent claims, and from the description which follows.

According to a first aspect of the present invention, there is provided a solid cleansing composition comprising:
(a) at least 35 wt % of one or more taurate surfactants of formula (I):

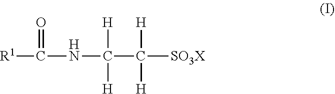

wherein X is hydrogen, a metal ion or an optionally substituted ammonium ion; and R¹ is an optionally substituted C₃-C₃₅ hydrocarbyl group; and
(b) at least 15 wt % of one or more fatty acids.

In some embodiments, the solid cleansing composition of the first aspect is a personal cleansing composition, suitably a personal cleansing bar.

In some embodiments, the solid cleansing composition of the first aspect is a laundry cleansing composition, suitably a laundry cleansing bar.

In some embodiments, the solid cleansing composition of the first aspect is a hard surface cleansing composition, suitably a hard surface cleansing bar.

For the avoidance of doubt, wherein amounts of components in a composition are described in wt %, this means the weight percentage of the specified component in relation to the whole composition referred to. Said weight percentages refer to the amount of the active, specified, component present in said composition, which may be referred to as the “weight % active” or “wt % active”. Said weight percentages do not include any solvent, water or impurities present, for example.

According to a second aspect of the present invention, there is provided a method of forming a solid cleansing composition according to the first aspect, the method comprising the steps of:
(i) forming a first surfactant composition by reacting an excess of one or more fatty acids of formula R¹COOH, or a reactive equivalent thereof, with a compound of formula (II):

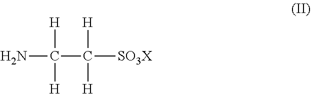

wherein X is hydrogen, a metal ion or an optionally substituted ammonium ion; and R¹ is an optionally substituted C₃-C₃₅ hydrocarbyl group;
wherein the first surfactant composition comprises:
   (a) at least 45 wt % of one or more taurate surfactants of formula (I):

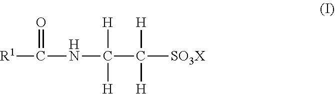

wherein X is hydrogen, a metal ion or an optionally substituted ammonium ion; and R¹ is an optionally substituted C₃-C₃₅ hydrocarbyl group; and
   (b) at least 20 wt % of the one or more fatty acids of formula R¹COOH;
and
(ii) combining the first surfactant composition formed in step (i) with at least one of:
   (c) at least one secondary surfactant, preferably selected from a non-ionic surfactant, a zwitterionic surfactant, a cationic surfactant or an anionic surfactant which is not a taurate surfactant of formula (I), or a mixture thereof; or
   (d) at least one additional ingredient selected from plasticizers, fragrances, dyes, opacifiers, pearlizers, fillers, chelating agents, pH adjustment agents, cationic polymers, conditioning agents, anti-cracking/hardening agents, anti-mushing/sloughing agents and active ingredients;
to form the solid cleansing composition.

By “excess of the one or more fatty acids of formula R¹COOH”, we mean that the molar equivalents of the one or more fatty acids, or reactive equivalents thereof, used in the reaction is greater than molar equivalents of the compound of formula (II) used in the reaction. The “excess” of fatty acid may be alternatively defined as greater than one molar equivalents of the fatty acid, compared to the compound of formula (II).

According to a third aspect of the present invention, there is provided a method of cleansing skin and/or hair, the method comprising contacting the skin and/or hair with a solid cleansing composition according to the first aspect.

According to a fourth aspect of the present invention, there is provided a use of a solid cleansing composition according to the first aspect for cleansing skin and/or hair.

According to a fifth aspect of the present invention, there is provided a cleansing product comprising a solid cleansing composition according to the first aspect and a packaging.

According to a sixth aspect of the present invention, there is provided a method of cleansing fabrics or hard surfaces, the method comprising contacting the fabric or hard surface with a solid cleansing composition according to the first aspect. The fabric cleansed by the method is suitably a soiled fabric. The hard surface cleansed by the method is suitably a soiled hard surface.

According to a seventh aspect of the present invention, there is provided a use of a solid cleansing composition according to the first aspect for cleansing fabrics or hard surfaces. The fabric cleansed by said use is suitably a soiled fabric. The hard surface cleansed by said use is suitably a soiled hard surface.

The solid cleansing compositions of the present invention comprise relatively large proportions of acyl taurate surfactants and fatty acids. This combination of components can provide the solid cleansing composition, for example as a syndet bar, with the desirable properties, such as hardness, foaming and wear, expected of a personal cleansing bar, suitably without using significant quantities of fatty acid soap components. This combination of components may also allow the solid cleansing composition to be made, for example in bar form, using conventional soap making techniques and equipment, which may provide cost and efficiency advantages compared to current processes for manufacturing syndet bars.

Both the acyl taurate surfactants of component (a) and the fatty acids of component (b) can be obtained with a 100% % RCI and therefore may allow the production of a solid cleansing composition, such as a syndet cleansing bar, which is considered completely natural and/or sustainably produced. Such a sustainably produced solid cleansing composition may have a significantly reduced environmental impact compared to known cleansing compositions, especially known syndet or combi bars.

Furthermore, the acyl taurate surfactants and fatty acids of the solid cleansing composition of the present invention can be provided together, for example as in the first surfactant composition, by a direct amidation reaction as described herein. Suitably the direct amidation reaction involves the formation of the amide bond of the acyl taurate surfactant by reacting greater than one molar equivalents of fatty acid with the taurine, such as a compound of formula (II). Suitably the acyl taurate surfactant is formed in situ to provide a first surfactant composition as described herein comprising the acyl taruate and the fatty acid, the fatty acid being provided by the unreacted excess of the fatty acid used in the reaction. The first surfactant composition formed by this reaction can be obtained as a solid, for example as a solid flake material, which facilitates the incorporation of the acyl taurate surfactants and fatty acids into a solid cleansing composition using conventional soap making techniques. This first surfactant composition can be directly incorporated in the solid cleansing compositions of the present invention without further processing, for example without purification or water removal, further contributing to the efficiency of the manufacturing process. The first surfactant composition may be provided as a molten liquid to which other ingredients of a solid cleansing composition can be added, to produce the solid cleansing composition of the present invention.

Preferred features of the first, second, third, fourth, fifth, sixth and seventh aspects will now be described.

The solid cleansing composition of the first aspect comprises (a) at least 35 wt % of one or more taurate surfactants of formula (I):

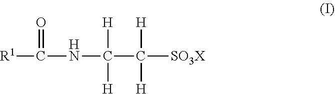

wherein X is hydrogen, a metal ion or an optionally substituted ammonium ion; and R¹ is an optionally substituted C₃-C₃₅ hydrocarbyl group.

Suitably component (a), the one or more taurate surfactants of formula (I), is present in an amount of at least 40 wt % of the solid cleansing composition, suitably at least 45 wt % or at least 50 wt %.

Suitably component (a) is present in an amount of up to 80 wt % of the solid cleansing composition, suitably up to 70 wt % or up to 65 wt %.

Suitably component (a) is present in an amount of from 35 to 80 wt % or from 40 to 80 wt % of the solid cleansing composition, suitably from 45 to 75 wt % or from 50 to 70 wt %.

X may be hydrogen, a metal ion or an optionally substituted ammonium ion. Suitable metal cations include alkali metal cations, for example sodium, lithium and potassium cations, and alkaline earth metal cations, for example calcium and magnesium cations. Suitably, X is an alkali metal cation or an optionally substituted ammonium cation. Preferably, X is a potassium or sodium cation. Most preferably, X is a sodium cation.

R¹ is an optionally substituted C₃-C₃₅ hydrocarbyl group. Suitably R¹ is an optionally substituted C₅-C₂₉ hydrocarbyl group, suitably an optionally substituted C₇-C₁₇ hydrocarbyl group.

Preferably R¹ is an optionally substituted C₃-C₃₅ alkyl or alkenyl group, an optionally substituted C₅-C₂₉ alkyl or alkenyl group or an optionally substituted C₇-C₁₇ alkyl or alkenyl group.

Preferably R¹ is a C₃-C₃₅ alkyl or alkenyl group, a C₅-C₂₉ alkyl or alkenyl group or a C₇-C₁₇ alkyl or alkenyl group.

Suitably R¹ is an unsubstituted C₃-C₃₅ alkyl or alkenyl group, an unsubstituted C₅-C₂₉ alkyl or alkenyl group or an unsubstituted C₇-C₁₇ alkyl or alkenyl group.

R¹ may be provided by a mixture of more than one of said groups.

R¹ is preferably an alkyl group or a mixture of alkyl groups with said number of carbons.

Preferably the R¹ groups of component (a) comprise less than 5 wt % alkenyl groups, suitably less than 1 wt % alkenyl groups, suitably substantially no alkenyl groups.

Preferably R¹ is an unsubstituted C₇-C₁₇ alkyl group.

R¹ is suitably the residue of a fatty acid. Fatty acids obtained from natural oils often include mixtures of fatty acids. For example, the fatty acid obtained from coconut oil contains a mixture of fatty acids including C₁₂ lauric acid, C₁₄ myristic acid, C₁₆ palmitic acid, C₈ caprylic acid, C₁₀ capric acid and C₁₈ stearic and oleic acid. Therefore, R¹ may be provided by the residues of a mixture of said fatty acids.

Preferably, R¹ is the residue of a hydrogenated fatty acid and therefore R¹ contains substantially no alkenyl groups. For example, R¹ may be provided by the residues resulting from the hydrogenation of coconut oil. Therefore, R¹ may be provided by the residues of a mixture of C₁₂ lauric acid, C₁₄ myristic acid, C₁₆ palmitic acid, C₈ caprylic acid, C₁₀ capric acid and C₁₈ stearic acid. R¹ may be a mixture of C₈, C₁₀, C₁₂, C₁₄, C₁₆ and C₁₈ alkyl groups, suitably straight chain alkyl groups, suitably derived from coconut oil. Fatty acids derived from coconut oil comprise at least 85 wt % fatty acids having 12 to 18 carbon atoms. Therefore, R¹ suitably comprises at least 85 wt % of C₁₂-C₁₈ alkyl groups. Suitably, in said mixtures, the majority of the R¹ groups are C₁₂ alkyl groups. R¹ may be provided by a topped fatty acid having a selected fatty acid chain length, wherein fatty acids with other chain lengths have been reduced or removed. For example, a topped fatty acid having predominantly C₁₂ alkyl groups as the R¹ groups may be used.

R¹ may include the residue of one or more naturally occurring fatty acids and/or of one or more synthetic fatty acids. Suitably R¹ is provided by one or more naturally occurring fatty acids.

In some embodiments R¹ may consist essentially of the residue of a single fatty acid.

Examples of fatty acids from which R¹ may be derived include coconut fatty acid, hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from rice bran oil, oat oil, wheat germ oil, hemp seed oil, coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof.

R¹ may be provided by the fatty acids obtained from coconut oil and/or palm kernel oil.

R¹ may be provided by lauric acid.

Preferably, component (a) is a cocoyl taurate or a lauroyl taurate, suitably sodium cocoyl taurate (SCT) or sodium lauroyl taurate.

Suitably component (a) has a percentage renewable carbon index (% RCI) of at least 70%, at least 80%, at least 90% or at least 98%, preferably at least 99%. Preferably component (a) has a % RCI of from 95 to 100%, preferably from 99 to 100%. Preferably component (a) has a % RCI of approximately 100%.

Suitably component (a) is a solid at room temperature. Suitably component (a) is in solid form when incorporated into the solid cleansing composition.

In some embodiments, component (a) may be in a molten, liquid form when mixed with other ingredients in the formation of the solid cleansing composition.

Component (a) may be provided by the reaction of one or more fatty acids of formula R₁COOH or a reactive equivalent thereof, with a compound of formula (II):

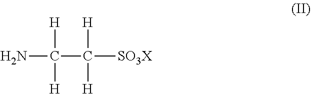

wherein X is hydrogen, a metal ion or an optionally substituted ammonium ion; and R¹ is an optionally substituted C₃-C₃₅ hydrocarbyl group. For example, component (a) may be provided by the reaction of the compound of formula (II) with an acid chloride derivative of such fatty acids or a methyl ester of such fatty acids. Component (a) may then be purified and combined with the fatty acid of component (b) in the production of the solid cleansing composition of this first aspect.

The solid cleansing composition of the first aspect comprises (b) at least 15 wt % of one or more fatty acids.

Suitably component (b), the one or more fatty acids, is present in an amount of at least 20 wt % of the solid cleansing composition, suitably at least 25 wt %.

Suitably component (b) is present in an amount of up to 60 wt % of the solid cleansing composition, suitably up to 50 wt % or up to 45 wt %.

Suitably component (b) is present in an amount of from 15 to 60 wt % of the solid cleansing composition, suitably from 20 to 50 wt %, from 15 to 50 wt %, from 25 to 50 wt % or from 25 to 45 wt %.

The fatty acid component (b) suitably has the formula R²COOH, wherein R² is as defined in relation to R¹ of component (a) of formula (I). Therefore, R² is an optionally substituted C₃-C₃₅ hydrocarbyl group. Suitably R¹ is an optionally substituted C₅-C₂₉ hydrocarbyl group, suitably a C₇-C₁₇ hydrocarbyl group.

Preferably R² is a C₃-C₃₅ alkyl or alkenyl group, a C₅-C₂₉ alkyl or alkenyl group or a C₇-C₁₇ alkyl or alkenyl group.

Suitably R² is an unsubstituted C₃-C₃₅ alkyl or alkenyl group, a C₅-C₂₉ alkyl or alkenyl group or a C₇-C₁₇ alkyl or alkenyl group.

Preferably R² is an unsubstituted C₇-C₁₇ alkyl group.

Component (b) is suitably provided by or derived from fatty acids obtained from a natural oil, which often include mixtures of fatty acids. Component (b) may be a fatty acid obtained from coconut oil which contains a mixture of fatty acids including C₁₂ lauric acid, C₁₄ myristic acid, C₁₆ palmitic acid, C₈ caprylic acid, C₁₀ capric acid and C₁₈ stearic and oleic acid. Therefore, component (b) may be a mixture of said fatty acids.

Preferably, component (b) is a hydrogenated fatty acid and therefore R² contains substantially no alkenyl groups. Component (b) may be produced by the hydrogenation of coconut oil. Therefore, component (b) may be provided by a mixture of C₁₂ lauric acid, C₁₄ myristic acid, C₁₆ palmitic acid, C₈ caprylic acid, C₁₀ capric acid and C₁₈ stearic acid. R² may be a mixture of C₈, C₁₀, C₁₂, C₁₄, C₁₆ and C₁₈ alkyl groups, suitably straight chain alkyl groups, suitably derived from coconut oil. Fatty acids derived from coconut oil comprise at least 85 wt % fatty acids having 12 to 18 carbon atoms. Therefore, component (b) suitably comprises at least 85 wt % of C₁₂-C₁₈ fatty acids. Suitably, the majority component (b) is provided by C₁₂ lauric acid.

Component (b) may comprise one or more naturally occurring fatty acids and/or of one or more synthetic fatty acids. Suitably component (b) is provided by one or more naturally occurring fatty acids.

In some embodiments R² may consist essentially of the residue of a single fatty acid.

Component (b) may be selected from coco acid (coconut fatty acid), hexanoic acid, caproic acid, caprylic acid, capric acid, lauric acid, myristic acid, palmitic acid, palmitoleic acid, stearic acid, oleic acid, linoleic acid, arachidic acid, gadoleic acid, arachidonic acid, eicosapentanoic acid, behenic acid, erucic acid, docosahexanoic lignoceric acid, naturally occurring fatty acids such as those obtained from rice bran oil, oat oil, wheat germ oil, hemp seed oil, coconut oil, tallow, palm kernel oil, butterfat, palm oil, olive oil, corn oil, linseed oil, peanut oil, fish oil and rapeseed oil; synthetic fatty acids made as chains of a single length or a selected distribution of chain lengths; and mixtures thereof.

Suitably component (b) comprises less than 10 wt % stearic acid, suitably less than 5 wt %. Suitably the solid cleansing composition comprises less than 10 wt % stearic acid, suitably less than 5 wt % or less than 3 wt % stearic acid.

In some embodiments, component (b) comprises fatty acids obtained from coconut oil and/or palm kernel oil. Suitably component (b) is provided by fatty acids obtained from coconut oil and/or palm kernel oil.

In some embodiments, component (b) comprises lauric acid. Suitably component (b) is lauric acid.

Preferably R² of component (b) is the same as R¹ of component (a).

Suitably component (b) has a percentage renewable carbon index (% RCI) of at least 70%, at least 80%, at least 90% or at least 98%, preferably at least 99%. Preferably component (a) has a % RCI of from 95 to 100%, preferably from 99 to 100%. Preferably component (b) has a % RCI of approximately 100%.

Suitably component (b) is a solid at room temperature. Suitably component (b) is in solid form when incorporated into the solid cleansing composition.

In some embodiments, component (b) may be in a molten, liquid form when mixed with other ingredients in the formation of the solid cleansing composition.

Suitably component (b) is provided together with component (a) in a first surfactant composition, as described below. Suitably said first surfactant composition is a solid at room temperature. Suitably said first surfactant composition comprising components (a) and (b) is in solid form when incorporated into the solid cleansing composition.

In some embodiments, the first surfactant composition comprising components (a) and (b) may be in a molten, liquid form when mixed with other ingredients in the formation of the solid cleansing composition.

In some embodiments, component (a) comprises a cocoyl taurate or a lauroyl taurate; and component (b) comprises coconut fatty acid or lauric acid. Suitably component (a) comprises sodium cocoyl taurate or sodium lauroyl taurate; and component (b) comprises coconut fatty acid or lauric acid.

In some embodiments, component (a) comprises sodium cocoyl taurate; and component (b) comprises coconut fatty acid. Suitably component (a) is sodium cocoyl taurate; and component (b) is coconut fatty acid.

In some embodiments, component (a) comprises sodium lauroyl taurate; and component (b) comprises lauric acid. Suitably component (a) is sodium lauroyl taurate; and component (b) is lauric acid.

Suitably component (a) is present in an amount of from 40 to 80 wt % and component (b) is present in an amount of from 15 to 50 wt %, in the solid cleansing composition of this first aspect.

Suitably components (a) and (b) together provide at least 70 wt % of the solid cleansing composition, suitably at least 80 wt % or at least 90 wt %.

Suitably component (a) and (b) are present in the solid cleansing composition in a weight ratio of from 4:1 to 1:2, suitably from 3:1 to 1:1, from 2:1 to 1:1 or from 2:1 to 1.5:1.

In some embodiment, the components (a) and (b) may be separately obtained/formed and combined to provide the solid cleansing composition of this first aspect.

In some embodiments, components (a) and (b) are provided together directly, i.e. in the same first surfactant composition, by the reaction of the compound of formula (II) with the one or more reactive derivatives of fatty acids of formula R¹COOH or a reactive equivalent thereof. In such embodiments, the reactive derivatives of fatty acids of formula R¹COOH or reactive equivalents thereof provide the component (b) fatty acid of formula R²COOH (and so R¹=R²).

Suitable reactive equivalents of fatty acids of formula R¹COOH include esters, anhydrides and acid chlorides of said fatty acids.

In such embodiments, components (a) and (b) may be provided by the reaction of the compound of formula (II) with said reactive derivatives of fatty acids of formula R¹COOH followed by hydrolysis of remaining reactive derivatives to provide the fatty acid of component (b) (wherein R²COOH=R¹COOH).

Alternatively, components (a) and (b) may be provided by the reaction of the compound of formula (II) with a fatty triglyceride comprising groups R¹, and suitably a catalyst, to provide component (a), followed by hydrolysis of unreacted or partially reacted fatty triglyceride to provide the fatty acid of component (b) (wherein R¹=R² of component (b)). Suitable fatty triglycerides include the oils referred to above, for example palm kernel oil or coconut oil.

In some embodiments of the solid cleansing composition of this first aspect, components (a) and (b) are provided by reaction of an excess of one or more fatty acids of formula R¹COOH or a reactive equivalent thereof, with a compound of formula (II):

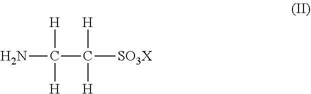

wherein X is hydrogen, a metal ion or an optionally substituted ammonium ion; and R¹ is an optionally substituted C₃-C₃₅ hydrocarbyl group and as further defined in relation to the R¹ group of component (a) of formula (I).

This reaction may be referred to as a direct amidation reaction, which forms an amide bond between the taurine compound of formula (II) and the fatty acid.

This reaction produces the taurate surfactant of formula (I) to provide component (a) and an unreacted excess of the fatty acid of formula R¹COOH provides component (b) (so R¹COOH=R²COOH, wherein R¹ of the taurate surfactant of formula (I) and R¹ of the fatty acid of component (b) are the same, both being derived from the one or more fatty acids of formula R¹COOH used in the reaction. The reaction suitably provides a first surfactant composition comprising components (a) and (b), suitably consisting essentially or consisting of components (a) and (b). In some embodiments, the first surfactant composition is a solid surfactant composition.

The one or more fatty acids of formula R¹COOH used in the reaction are suitably as described above in relation to R²COOH of component (b).

This reaction directly provides components (a) and (b), suitably in the relative proportions discussed above. The reaction suitably provides the combination of components (a) and (b) in solid form which is readily handleable in subsequent processing to form a solid cleansing composition according to the first aspect of the present invention. For example, the combination of components (a) and (b) may be obtained from the reaction as solid flakes. Said flakes of components (a) and (b) can be combined with the further components discussed above to form a solid cleansing composition, suitably using conventional soap making techniques. Alternative methods for forming the taurate surfactant of formula (I) may provide a liquid or paste form which is difficult to process into a solid cleansing composition, for example in bar form. Such liquids or pastes would require further processing steps in order to be successfully incorporated into a solid cleansing composition, for example a personal cleansing bar.

In some embodiments, component (a) and a first portion of component (b) are provided together directly, i.e. in the same first surfactant composition, by a reaction as discussed above, e.g. a reaction of a compound of formula (II) with one or more reactive derivatives of fatty acids of formula R¹COOH or a reactive equivalent thereof. In such embodiments, a second portion of component (b) is suitably added to a mixture of component (a) and the first portion of component (b).

The second portion of component (b) may be the same fatty acid or mixture of fatty acids as the first portion of component (b). For example, the first portion of component (b) may be provided by unreacted excess coconut fatty acid from the reaction used to form component (a) (as described herein), and the second portion of component (b) may be provided by additional coconut fatty acid added to component (a) and the first portion of component (b).

The second portion of component (b) may be a different fatty acid or mixture of fatty acids to the first portion of component (b). Therefore the first portion of component (b) may be a first fatty acid or fatty acid mixture and the second portion of component (b) may be a second fatty acid or fatty acid mixture. For example, the first portion of component (b) may be provided by unreacted excess coconut fatty acid from the reaction used to form component (a) (as described herein), and the second portion of component (b) may be provided by stearic acid added to component (a) and the first portion of component (b).

The solid cleansing composition of this first aspect may comprise component (c) at least 1 wt % of at least one secondary surfactant. Such a secondary surfactant is in addition to the taurate surfactant of component (a). Therefore, component (c) is suitably not a taurate surfactant of formula (I). Suitably component (c) is selected from a non-ionic surfactant, a zwitterionic surfactant, a cationic surfactant or an anionic surfactant which is not a taurate surfactant of formula (I), or a mixture thereof.

Suitably component (c) is present in an amount of at least 2 wt % of the solid cleansing composition, suitably at least 3 wt %.

Suitably component (c) is present in an amount of up to 10 wt % of the solid cleansing composition, suitably up to 8 wt % or up to 6 wt %.

Suitably component (c) is present in an amount of from 1 to 10 wt % of the solid cleansing composition, suitably from 2 to 8 wt % or from 3 to 6 wt %.

Suitably components (a), (b) and (c) together provide at least 75 wt % of the solid cleansing composition, suitably at least 85 wt % or at least 90 wt %.

Suitable anionic surfactants for use herein include salts of: fatty acids; alkoxylated carboxylic acids; ester carboxylates; ethoxylated ester carboxylates; mono- or dialkyl sulfates; mono- or dialkyl ether sulfates; lauryl ether sulfates; alkyl sulfonates; alkyl aryl sulfonates; primary alkane disulfonates; alkene sulfonates; hydroxyalkane sulfonates; isethionates, alkyl isethionates, acyl isethionates, acyl alkyl isethionates, alkyl acyl taurates, alkyl glyceryl ether sulfonates; alpha-olefin sulfonates; alkylphosphates; sulfonates of alkylphenolpolyglycol ethers; alkyl sulfopolycarboxylic acid esters; alkyl sulfosuccinates; alkyl ether sulfosuccinates; products of condensation of fatty acids with oxy- and aminoalkanesulfonic acids; sulfated derivatives of fatty acids and polyglycols; alkyl and acyl sarcosinates; sulfoacetates; alkyl phosphates; alkylphosphate esters; acyl lactates; alkanolamides of sulfated fatty acids, lipoamino acids and acyl substituted amino acids, for example acyl glycinates and acyl glutamates.

Preferred anionic surfactants are selected from salts of fatty acids; alkyl sulfonates; alkyl aryl sulfonates; primary alkane disulfonates; alkene sulfonates; hydroxyalkane sulfonates; alkyl glyceryl ether sulfonates; alpha-olefin sulfonates; alkyl phosphates; sulfonates of alkylphenolpolyglycol ethers; salts of alkyl sulfopolycarboxylic acid esters; alkyl sulfosuccinates and salts thereof, alkyl ether sulfosuccinates and salts thereof, acyl isethionates, acyl alkyl isethionates, non-acylated alkyl isethionates; amino acid surfactants such as acyl and alkyl glutamates, acyl and alkyl sarcosinates, acyl and alkyl glycinates, acyl and alkyl alaninates and acyl and alkyl aspartates; products of condensation of fatty acids with oxy- and aminoalkanesulfonic acids; alkyl sulfoacetates; alkyl phosphates; alkyl phosphate esters; acyl lactylates; salts of lipoamino acids and biosurfactants, for example sophorolipids, rhamnolipids, saponins and surfactin.

Particularly exemplary salts of the above, where applicable, are the sodium, potassium, ammonium, magnesium and triethanolamine salts. Suitable ammonium cations include those derived from alkyl amines and alkanolamines. Preferred ammonium cations include isopropanolamine, isopropylamine, ethanolamine, diethanolamine, triethanolamine and 2-amino-2-methyl-1,3-propanediol (AMPD). Preferred ammonium cations include NH₄⁺ and the ammonium cation of triethanolamine.

Suitable sulfoacetates include acyl sulfoacetates, particularly sodium acyl sulfoacetates.

Suitable glutamate surfactants include monosodium or disodium acyl glutamates.

Suitable glycinate surfactants include monosodium acyl glycinates.

Suitable alaninate surfactants include monosodium acyl alaninates.

Suitable aspartate surfactants include monosodium or disodium acyl aspartates.

Preferred anionic surfactants include alkyl glyceryl ether sulfonate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, and combinations thereof.

In some embodiments, the secondary surfactant is provided by at least one or more acyl isethionate, acyl alkyl isethionate or acyl alkyl taurate surfactants, or mixtures thereof.

In embodiments wherein the secondary surfactant comprises an isethionate surfactant, the isethionate surfactant is suitably a compound of formula (III):

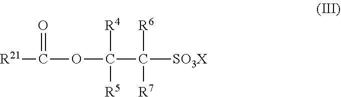

wherein R²¹ is as defined above for R¹ of component (a) of formula (I); each of R⁴, R⁵, R⁶ and R⁷ independently represents a hydrogen atom or a C₁₋₄ alkyl group; and X is as defined above for the X group of component (a) of formula (I). In some embodiments, R²¹ of the compound of formula (III) is the same as R¹ of component (a) of formula (I).

In some embodiments R⁴ represents a C₁₋₄ alkyl group, suitably a C₁₋₄ alkyl group in which a propyl or butyl group, when present, is straight-chained. Suitably R⁴ may represent an n-propyl, ethyl or preferably, a methyl group. In preferred embodiments R⁴ is hydrogen.

Preferably R⁵ represents a hydrogen atom.

In some embodiments one of R⁶ and R⁷ represent a hydrogen atom and the other represents a hydrogen atom or a C₁₋₄ alkyl group. Suitably one of R⁶ and R⁷ represents a hydrogen atom or a C₁₋₄ alkyl group in which a propyl or butyl group is straight-chain. Preferably one of R⁶ and R⁷ represents an n-propyl, ethyl or methyl group or, most preferably, a hydrogen atom. Most preferably both R⁶ and R⁷ represent hydrogen atoms.

In some embodiments each of R⁴, R⁵, R⁶ and R⁷ is hydrogen and the isethionate compound is of formula R²¹CO₂CH₂CH₂SO₃X.

Preferably X represents a potassium cation, or, especially, a sodium cation.

In some embodiments the isethionate surfactant may be a mixture of more than one compound of formula (III).

Suitably, the isethionate surfactant is selected from one or more of sodium lauroyl isethionate, sodium cocoyl isethionate, sodium lauroyl methyl isethionate, sodium cocoyl methyl isethionate and sodium oleoyl methyl isethionate.

In some embodiments, the isethionate surfactant is an acyl isethionate, suitably selected from sodium lauroyl isethionate or sodium cocoyl isethionate, or a mixture thereof.

In some embodiments, the isethionate surfactant may be an acyl alkyl isethionate, suitably selected from sodium lauroyl methyl isethionate, sodium cocoyl methyl isethionate and sodium oleoyl methyl isethionate, or a mixture thereof. Suitably the acyl alkyl isethionate is selected from sodium lauroyl methyl isethionate and sodium cocoyl methyl isethionate, or a mixture thereof.

In embodiments wherein the secondary surfactant comprises an acyl alkyl taurate surfactant, the acyl alkyl taurate surfactant is suitably a compound of formula (IV):

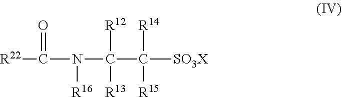

wherein X and R²² are suitably as defined above in relation to R¹ and X of component (a) of formula (I); wherein each of R¹², R¹³, R¹⁴ and R¹⁵ independently represents hydrogen or an optionally substituted C₁-C₄ alkyl group; and R¹⁶ is an optionally substituted C₁-C₄ alkyl group. In some embodiments, R²² of the compound of formula (IV) is the same as R¹ of component (a) of formula (I).

Suitable C₁ to C₄ alkyl groups for R¹², R¹³, R¹⁴, R¹⁵ and R¹⁶ may be selected from n-propyl, ethyl or methyl, such as ethyl or methyl, most preferably methyl.

In some embodiments R¹⁶ is methyl.

In some embodiments, each of R¹², R¹³, R¹⁴ and R¹⁵ is hydrogen and the compound of formula (IV) is an acyl N-alkyl taurate surfactant. In such embodiments R¹⁶ is preferably n-propyl, ethyl or methyl. Preferably R¹⁶ is ethyl or methyl, most preferably methyl. Thus in such embodiments the compound of formula (IV) is preferably an acyl N-methyl taurate surfactant.

In some embodiments one of the groups R¹², R¹³, R¹⁴ and R¹⁵ represents an optionally substituted C₁-C₄ alkyl group and the remaining groups represent hydrogen. For example, R¹² may represent an optionally substituted C₁-C₄ alkyl group and R¹³, R¹⁴ and R¹⁵ may all represent hydrogen. For example, R¹⁴ may represent an optionally substituted C₁-C₄ alkyl group and R¹², R¹³ and R¹⁵ may all represent hydrogen.

Preferably R¹² represents a methyl group and R¹³, R¹⁴ and R¹⁵ all represent hydrogen or R¹⁴ represents a methyl group and R¹², R¹³ and R¹⁵ all represent hydrogen.

The acyl alkyl taurate surfactant may include a mixture of more than one taurate compound of formula (IV). For example, an isomeric mixture of acyl N-alkyl alkyl taurate compounds may be present. Such a mixture may include, for example an acyl N-alkyl alkyl taurate compound in which R¹² represents a C₁-C₄ alkyl group (suitably methyl) and R¹³, R¹⁴ and R¹⁵ are all hydrogen and an acyl N-alkyl alkyl taurate compound in which R¹⁴ represents a C₁-C₄ alkyl group (suitably methyl) and R¹², R¹³ and R¹⁵ are all hydrogen.

Suitably such mixtures comprise at least 90% of compounds in which R¹² is methyl and R¹⁴ is hydrogen and at most 10% of compounds in which R¹² is hydrogen and R¹⁴ is methyl.

Preferably the acyl alkyl taurate surfactant comprises one or more taurate compounds of formula (IV) selected from sodium lauroyl methyl taurate, sodium cocoyl methyl taurate, sodium oleoyl methyl taurate, sodium myristoyl methyl taurate, sodium lauroyl N-methyl methyl taurate, sodium cocoyl N-methyl methyl taurate, sodium oleoyl N-methyl methyl taurate and sodium myristoyl N-methyl methyl taurate. Sodium lauroyl methyl taurate and sodium cocoyl methyl taurate are especially preferred.

In such embodiments wherein the secondary surfactant is provided by at least one or more acyl isethionate, acyl alkyl isethionate or acyl alkyl taurate surfactants, the secondary surfactant (c) is suitably selected from sodium lauroyl isethionate, sodium cocoyl isethionate, sodium lauroyl methyl isethionate, sodium cocoyl methyl isethionate sodium lauroyl methyl taurate and sodium cocoyl methyl taurate, or mixtures thereof.

In some embodiments, the solid cleansing composition of this first aspect comprises:
(c) at least 1 wt % of a secondary surfactant selected from:
   (c1) an isethionate surfactant of formula (III):

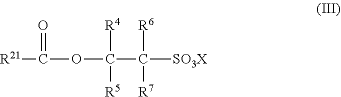

wherein R²¹ is as defined above for R¹ of component (a) of formula (I); each of R⁴, R⁵, R⁶ and R⁷ independently represents a hydrogen atom or a C₁₋₄ alkyl group; and X is as defined above for the X group of component (a) of formula (I); and
   (c2) an acyl alkyl taurate surfactant of formula (IV):

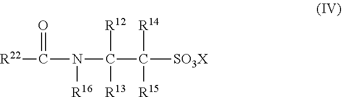

wherein X and R²² are as defined above in relation to R¹ and X of component (a) of formula (I); wherein R¹², R¹³, R¹⁴ and R¹⁵ are each independently selected from hydrogen or an optionally substituted C₁-C₄ alkyl group; and R¹⁶ is an optionally substituted C₁-C₄ alkyl group;
or a mixture thereof.

Preferably, R², R²¹ and R²² are the same as R¹ of component (a) of formula (I).

Components (c1) and (c2) may be as defined above for the acyl isethionate, acyl alkyl isethionate or acyl alkyl taurate surfactants.

In some embodiments, component (a) comprises sodium cocoyl taurate; component (b) comprises coconut fatty acid; and the secondary surfactant component (c) comprises sodium cocoyl methyl taurate. Suitably component (a) is sodium cocoyl taurate; component (b) is coconut fatty acid; and component (c) is sodium cocoyl methyl taurate.

In some embodiments, component (a) comprises sodium lauroyl taurate; component (b) comprises lauric acid; and component (c) comprises sodium lauroyl methyl taurate. Suitably component (a) is sodium lauroyl taurate; component (b) is lauric acid; and component (c) is sodium lauroyl methyl taurate.

In some embodiments, component (a) comprises sodium cocoyl taurate; component (b) comprises coconut fatty acid; and the secondary surfactant component (c) comprises sodium cocoyl methyl isethionate. Suitably component (a) is sodium cocoyl taurate; component (b) is coconut fatty acid; and component (c) is sodium cocoyl methyl isethionate.

In some embodiments, component (a) comprises sodium lauroyl taurate; component (b) comprises lauric acid; and component (c) comprises sodium lauroyl methyl isethionate. Suitably component (a) is sodium lauroyl taurate; component (b) is lauric acid; and component (c) is sodium lauroyl methyl isethionate.

Suitably components (c1) and/or (c2) are present in an amount of at least 2 wt % of the solid cleansing composition, suitably at least 3 wt %.

Suitably components (c1) and/or (c2) are present in an amount of up to 10 wt % of the solid cleansing composition, suitably up to 8 wt % or up to 6 wt %.

Suitably components (c1) and/or (c2) are present in an amount of from 1 to 10 wt % of the solid cleansing composition, suitably from 2 to 8 wt % or from 3 to 6 wt %.

Suitably components (c1) and (c2) are present in a combined amount of at least 2 wt % of the solid cleansing composition, suitably at least 3 wt %.

Suitably components (c1) and (c2) are present in a combined amount of up to 10 wt % of the solid cleansing composition, suitably up to 8 wt % or up to 6 wt %.

Suitably components (c1) and (c2) are present in a combined amount of from 1 to 10 wt % of the solid cleansing composition, suitably from 2 to 8 wt % or from 3 to 6 wt %.

Suitably components (a), (b), (c1) and/or (c2) together provide at least 75 wt % of the solid cleansing composition, suitably at least 85 wt % or at least 90 wt %.

In some embodiments, the solid cleansing composition comprises both secondary surfactants (c1) and (c2). In such embodiments, the amounts of component (c1) and/or (c2) present in the solid cleansing composition referred to above relates to the total amount of secondary surfactants (c1) and (c2) present.

In such embodiments, the weight ratio of the amount of component (a) to the amount of components (c1) and (c2), taken together, in the solid cleansing composition is from 20:1 to 2:1, suitably from 10:1 to 2:1 or from 5:1 to 3:1, or from 2.5:1 to 1:1.

Suitably the weight ratio of the amount of component (c1) to the amount of component (c2) in the solid cleansing composition, is from 20:1 to 1:20, suitably from 10:1 to 1:10 or from 5:1 to 1:5.

Suitable non-ionic surfactants include alcohol alkoxylates such as alcohol ethoxylates, castor oil ethoxylates, alcohol propoxylates, and ethylene oxide/propylene oxide copolymer derived surfactants, aliphatic esters, aromatic esters, sugar esters, especially sorbitan esters, alkyl polyglucosides, N-acylglucamides, fatty acid alkoxylates such as fatty acid ethoxylates and fatty acid propoxylates or polyethylene glycol esters and partial esters, glycerol esters including glycerol partial esters and glycerol triesters, fatty alcohols (such as cetearyl alcohol, lauryl alcohol, stearyl alcohol, behenyl alcohol), alkanolamides, amine oxides and biosurfactants.

Suitable biosurfactants include rhamnolipids, trehalolipids, sophorolipids, mannosylerythritol lipids, and glycolipids produced by Meyerozama guilliermondii, Saccharomyces cerevisiae, Candida utilis, Candida bombicola and/or Marinobacter hydrocarbonoclasticus.

Preferred biosurfactants are selected from one or more of a rhamnolipid, a trehalolipid, a sophorolipid and/or a mannosylerythritol lipid.

Sophorolipids are especially preferred.

Suitable non-ionic surfactants may be selected from the following: reaction products of compounds having a hydrophobic group and a reactive hydrogen atom, for example aliphatic alcohols, acids, amides or alkyl phenols with alkylene oxides, especially ethylene oxide either alone or with propylene oxide (for example alkyl (C₆-C₂₂) phenol-ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine); long chain tertiary amine oxides, long chain tertiary phosphine oxides and dialkyl sulfoxides; alkyl amine oxides, alkyl amido amine oxides; alkyl tertiary phosphine oxides; alkoxyl alkyl amines; sorbitan; sorbitan esters; sorbitan ester alkoxylates; glycerol ester alkoxylates; sucrose esters; sugar amides, such as N-acylglucamides or polysaccharide amides; lactobionamides; alkyl polysaccharide nonionic surfactants, for example alkylpolyglycosides and alkyl polypentosides; and biosurfactants from fermentation processes, for example sophorolipids (lactonic form or acidic form) and mannosylerythritol lipids.

Suitable cationic surfactants are typically based on fatty amine derivates or phosphonium quaternary ions, and quaternary ammonium compounds. Polymeric cationic surfactants may also be used.

Suitable cationic surfactants for use in the present invention include tertiary amine salts, mono alkyl trimethyl ammonium chloride, mono alkyl trimethyl ammonium methyl sulfate, dialkyl dimethyl ammonium chloride, dialkyl dimethyl ammonium methyl sulfate, trialkyl methyl ammonium chloride and trialkyl methyl ammonium methyl sulfate.

Examples of suitable cationic surfactants include quaternary ammonium compounds, particularly fatty-alkyl trimethyl quaternary compounds, difatty-alkyl dimethyl quaternary compounds and ester quaternary compounds.

Preferred quaternary ammonium compounds include cetyltrimethylammonium chloride, behenyltrimethylammonium chloride (BTAC), cetylpyridinium chloride, tetramethylammonium chloride, tetraethylammonium chloride, octyltrimethylammonium chloride, chloride, hexadecyltrimethylammonium chloride, dodecyltrimethylammonium octyldimethylbenzylammonium chloride, decyldimethylbenzylammonium chloride, stearyldimethylbenzylammonium chloride, didodecyldimethylammonium chloride, dioctadecyldimethylammonium chloride, tallowtrimethylammonium chloride, cocotrimethylammonium chloride, PEG-2 oleylammonium chloride, ditallow dimethyl ammonium chloride, dicetyl dimethyl ammonium chloride, distearyl dimethyl ammonium chloride, ester quaternary ammonium compounds (examples of which are mentioned in J. Oleo Science, 56 (6), 269, (2007)) and salts of these where the chloride is replaced by halogen (e.g. bromide), acetate, citrate, lactate, glycolate, phosphate, nitrate, sulfate, alkylsulfate or alkyl sulfonate.

Further suitable cationic surfactants include those materials having the CTFA designations Quaternium-5, Quaternium-31 and Quaternium-18. Mixtures of any of the foregoing materials may also be suitable. A particularly useful cationic surfactant for use as a hair conditioning agent is cetyltrimethylammonium chloride, available commercially, for example as GENAMIN CTAC, Clariant.

Salts of primary, secondary, and tertiary fatty amines are also suitable cationic surfactants. The alkyl groups of such amines preferably have from 12 to 22 carbon atoms, and can be optionally substituted.

Useful cationic surfactants in this group include amido substituted tertiary fatty amines, in particular tertiary amines having one C₁₂ to C₂₂ alkyl or alkenyl chain. Such amines include stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachid amidopropyldiethylamine, arachidamidoethyldiethylamine, arachidamidoethyldimethylamine, diethylaminoethylstearamide.

Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidyl behenylamine.

These amines are typically used in combination with an acid to provide the cationic species. Suitable acids include L-glutamic acid, lactic acid, hydrochloric acid, malic acid, succinic acid, acetic acid, fumaric acid, tartaric acid, citric acid, L-glutamic hydrochloride, and mixtures thereof; more preferably L-glutamic acid, lactic acid, citric acid.

Other useful cationic amine surfactants include those disclosed in U.S. Pat. No. 4,275,055.

Suitable polymeric cationic surfactants include polyquaternium-7, polyquaternium-10, polyquaternium-11, guar hydroxypropyltrimonium chloride, and hydroxypropyl guar hydroxypropyltrimonium chloride.

Suitable amphoteric surfactants include those based on fatty nitrogen derivates and those based on betaines.

Suitable amphoteric or zwitterionic surfactants may be selected from betaines, for example alkyl betaines, alkylamidopropyl betaines, for example cocamidopropyl betaine, alkylamidopropyl hydroxy sultaines, alkylamphoacetates, alkylamphodiacetates, alkyl propionates, alkylamphodipropionates, alkylamphopropionates, alkyliminodipropionates, alkyliminodiacetate, dimethyl amine oxides and alkanoyl amidopropyldimethylamine oxides.

Amphoteric or zwitterionic surfactants for use may include those which have an alkyl or alkenyl group of 7 to 22 carbon atoms and comply with an overall structural formula:

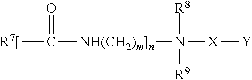

where R⁷ is alkyl or alkenyl of 7 to 22 carbon atoms; R⁸ and R⁹ are each independently alkyl, hydroxyalkyl or carboxyalkyl of 1 to 6 carbon atoms; m is 2 to 4; n is 0 or 1; X is alkylene of 1 to 6 carbon atoms optionally substituted with hydroxyl; and Y is —CO₂⁻ or —SO₃⁻.

Amphoteric or zwitterionic surfactants may include simple betaines of formula:

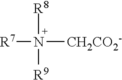

and amido betaines of formula:

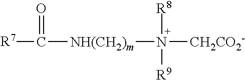

where m is 2 or 3.

In both formulae R⁷, R⁸ and R⁹ are as defined previously. R⁷ may, in particular, be a mixture of C₁₂ and C₁₄ alkyl groups derived from coconut so that at least half, preferably at least three quarters, of the groups R⁷ has 10 to 14 carbon atoms. R⁸ and R⁹ are preferably methyl.

Amphoteric or zwitterionic surfactants may include sulfobetaines of formula:

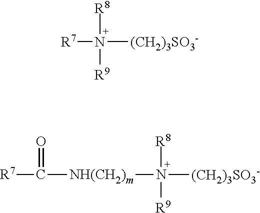

where m is 2 or 3, or variants of these in which
(CH₂)₃SO₃⁻ is replaced by

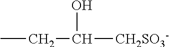

where R⁷, R⁸ and R⁹ in these formulae are as defined previously.

Amphoteric or zwitterionic surfactants may include amphoacetates and diamphoacetates. Amphoacetates generally conform to the following formula:

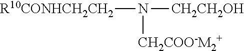

Diamphoacetates generally conform to the following formula:

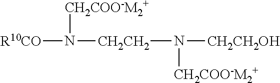

where R¹⁰ is an aliphatic group of 8 to 22 carbon atoms and M₂⁺ is a cation such as sodium, potassium, ammonium, or substituted ammonium.

Suitable acetate-based surfactants include lauroamphoacetate; alkyl amphoacetate; sodium alkyl amphoacetate; cocoampho (di) acetate; cocoamphoacetate; disodium cocoamphodiacetate; sodium cocoamphoacetate; disodium cocoamphodiacetate; disodium capryloamphodiacete; disodium lauroamphoacetate; sodium lauroamphoacetate and disodium wheatgermamphodiacetate.

Suitable betaine surfactants include alkylamido betaine; alkyl betaine, C_{12/14} alkyldimethyl betaine; cocoamidopropylbetaine; tallow bis(hydroxyethyl) betaine; hexadecyldimethylbetaine; cocodimethylbetaine; alkyl amido propyl sulfo betaine; alkyl dimethyl amine betaine; coco amido propyl dimethyl betaine; alkyl amido propyl dimethyl amine betaine; cocamidopropyl betaine; lauryl betaine; laurylamidopropl betaine, coco amido betaine, lauryl amido betaine, alkyl amino betaine; alkyl amido betaine; coco betaine; lauryl betaine; dimethicone propyl PG-betaine; oleyl betaine; N-alkyldimethyl betaine; coco biguamide derivative, C₈ amido betaine; C₁₂ amido betaine; lauryl dimethyl betaine; alkylamide propyl betaine; amido betaine; alkyl betaine; cetyl betaine; oleamidopropyl betaine; isostearamidopropyl betaine; lauramidopropyl betaine; 2-alkyl-N-carboxymethyl-N-hydroxyethyl imidazolinium betaine; 2-alkyl-N-carboxyethyl-N-hydroxyethyl imidazolinium betaine; 2-alkyl-N-sodium carboxymethyl-N-carboxymethyl oxyethyl imidazolinium betaine; N-alkyl acid amidopropyl-N,N-dimethyl-N-(3-sulfopropyl)-ammonium-betaine; N-alkyl-N,N-dimethyl-N-(3-sulfopropyl)-ammonium-betaine; cocodimethyl betaine; apricotamidopropyl betaine; isostearamidopropyl betaine; myristamidopropyl betaine; palmitamidopropyl betaine; alkamidopropyl hydroxyl sultaine; cocamidopropyl hydroxyl sultaine; undecylenamidopropyl betaine; cocoamidosulfobetaine; alkyl amido betaine; C_{12/18} alkyl amido propyl dimethyl amine betaine; lauryldimethyl betaine; ricinol amidobetaine; tallow aminobetaine.

Suitable amphoteric glycinate surfactants include acyl glycinates such as cocoamphocarboxyglycinate; tallowamphocarboxygycinate; capryloamphocarboxyglycinate, oleoamphocarboxyglycinate, bis-2-hydroxyethyl tallow glycinate; lauryl amphoglycinate; tallow polyamphoglycinate; coco amphoglycinate; oleic polyamphoglycinate; N—C_{10/12} fatty acid amidoethyl-N-(2-hydroxyethyl)-glycinate; N—C_{12/18}-fatty acid amidoethyl-N-(2-hydroxyethyl)-glycinate; dihydroxyethyl tallow gycinate.

Preferred acetate-based amphoteric surfactants include sodium lauroamphoacetate, disodium lauroamphoacetate and mixtures thereof.

Preferred betaine surfactants include cocoamidopropyl betaine.

Preferred sultaine surfactants include cocoamidopropylhydroxy sultaine.

In some embodiments, the solid cleansing composition may comprise fatty acid soap. Such fatty acid soaps may be simply referred to as “soaps” and are salts of fatty acids. Fatty acid soap is a type of anionic surfactant. In such embodiments, the fatty acid soap may provide component (c) the secondary surfactant. In such embodiments, the fatty acid soap of component (c) is distinct from the fatty acid of component (b), the fatty acid soap being a salt of a fatty acid and component (b) being a free acid (i.e. in neutral form).

A fatty acid soap may include the alkali metal or alkanol ammonium salts of aliphatic alkane or alkene monocarboxylic acids. Sodium, potassium, mono-, di- and tri-ethanol ammonium cations, or combinations thereof, are suitable for use in the solid cleansing composition. In general, sodium fatty acid soap may be used in the solid cleansing compositions of this invention, but from about 1 wt % to about 25 wt % of the fatty acid soap may be potassium fatty acid soap. The soap component useful herein may include the well-known alkali metal salts of natural of synthetic aliphatic (alkanoic or alkenoic) acids having about 12 to 22 carbon atoms, preferably about 12 to about 18 carbon atoms. They may be described as alkali metal carboxylates of acyclic hydrocarbons having about 12 to about 22 carbon atoms.

Fatty acid soaps having a range of molecular weights may be used. Fatty acid soap having the fatty acid distribution of coconut oil may provide the lower end of the broad molecular weight range. Those soaps having the fatty acid distribution of peanut or rapeseed oil, or their hydrogenated derivatives, may provide the upper end of the broad molecular weight range.

It is preferred to use fatty acid soaps having the fatty acid distribution of coconut oil or palm kernel oil, or mixtures thereof. The proportion of fatty acids having at least 12 carbon atoms in coconut oil soap is about 85%. This proportion will be greater when mixtures of coconut oil and fats such as tallow, palm oil, or non-tropical nut oils or fats are used, wherein the principal carbon chain lengths are C16 and higher. Preferred soaps for use in the present invention have at least about 85% fatty acids having about 12 to 18 carbon atoms.

Coconut oil employed for the soap component may be substituted in whole or in part by other “high-alluric” oils, that is, oils or fats wherein at least 50% of the total fatty acids are composed of lauric or myristic acids and mixtures thereof. These oils are generally exemplified by the tropical nut oils of the coconut oil class. For instance, they include: palm kernel oil, babassu oil, ouricuri oil, tucum oil, cohune nut oil, murumuru oil, jaboty kernel oil, khakan kernel oil, dika nut oil, and ucuhuba butter.

A preferred fatty acid soap component is a mixture of about 15% to about 20% coconut oil and about 80% to about 85% tallow. These mixtures contain about 95% fatty acids having about 12 to about 18 carbon atoms. The soap component may be prepared from coconut oil, in which case the fatty acid content is about 85% of C12-C18 chain length.

The fatty acid soap may contain unsaturation in accordance with commercially acceptable standards. Excessive unsaturation is normally avoided.

The fatty acid soap component may be made by the classic kettle boiling process or modern continuous soap manufacturing processes wherein natural fats and oils such as tallow or coconut oil or their equivalents are saponified with an alkali metal hydroxide using procedures well known to those skilled in the art. Alternatively, the additional soaps may be made by neutralizing fatty acids, such as lauric (C12), myristic (C14), palmitic (C16), or stearic (C18) acids with an alkali metal hydroxide or carbonate.

Suitably, the fatty acid soap has the formula: R³COOX wherein X is an alkali metal ion; and R³ is an optionally substituted C₃-C₃₅ hydrocarbyl group. Suitably R³ is as defined in relation to R¹ of component (a). Suitably R³ of the fatty acid soap is the same as R¹ of component (a) and/or the same as R² of component (b). Suitably R¹, R² and R³ are the same, suitably provided by the fatty acids obtained from coconut oil and/or palm kernel oil or provided by lauric acid.

Suitably, the fatty acid soap is an alkali metal salt of coconut fatty acid or lauric acid. Suitably the fatty acid soap is sodium cocoate or sodium laurate.

In some embodiments, component (a) comprises a cocoyl taurate or a lauroyl taurate; component (b) comprises coconut fatty acid or lauric acid; and the fatty acid soap of component (c) comprises an alkali metal salt of coconut fatty acid or lauric acid.

Suitably component (a) comprises sodium cocoyl taurate or sodium lauroyl taurate; component (b) comprises coconut fatty acid or lauric acid; and the fatty acid soap comprises sodium cocoate or sodium laurate.

In some embodiments, component (a) comprises sodium cocoyl taurate; component (b) comprises coconut fatty acid; and the fatty acid soap of component (c) comprises sodium cocoate. Suitably component (a) is sodium cocoyl taurate; component (b) is coconut fatty acid; and the fatty acid soap of component (c) is sodium cocoate.

In some embodiments, component (a) comprises sodium lauroyl taurate; component (b) comprises lauric acid; and the fatty acid soap of component (c) comprises sodium laurate. Suitably component (a) is sodium lauroyl taurate; component (b) is lauric acid; and the fatty acid soap of component (c) is sodium laurate.

Suitably the solid cleansing composition according to this first aspect comprises less than 15 wt % of fatty acid soap, suitably less than 10 wt % or less than 5 wt %.

Suitably the fatty acid soap is present in an amount of from 1 to 8 wt % of the solid cleansing composition, suitably from 2 to 8 wt % or from 3 to 6 wt %.

In such embodiments, the solid cleansing composition in bar form may be considered a combi bar due to the composition comprising such amounts of fatty acid soap. Alternatively, the solid cleansing composition in bar form may be considered a syndet bar due to the fatty acid soap being present in a relatively low proportion compared to the taurate surfactant.

In some embodiments, the solid cleansing composition does not comprise any fatty acid soap. Such solid cleansing compositions may be referred to as “soap free”. Such soap free solid cleansing compositions suitably contain less than 5 wt % of a fatty acid soap as defined herein, preferably less than 3 wt %, more preferably less than 1 wt %, preferably less than 0.1 wt % and most preferably less than 0.01 wt %.

In such embodiments the solid cleansing composition in bar form may be considered a syndet bar.

In some embodiments, the solid cleansing composition comprises:
(a) at least 35 wt % of one or more of sodium cocoyl taurate or sodium lauroyl taurate; and
(b) at least 15 wt % of one or more of coconut fatty acid or lauric acid;
wherein the composition comprises less than 15 wt % of fatty acid soap, preferably less than 1 wt % fatty acid soap.

In some embodiments, the solid cleansing composition comprises:
(a) at least 50 wt % of one or more of sodium cocoyl taurate or sodium lauroyl taurate; and
(b) at least 25 wt % of one or more of coconut fatty acid or lauric acid;
wherein the composition comprises less than 10 wt % of fatty acid soap, preferably less than 1 wt % fatty acid soap.

The solid cleansing composition of this first aspect may comprise component (d) at least 1 wt % of at least one additional ingredient selected from plasticizers, fragrances, dyes, opacifiers, pearlizers, fillers, chelating agents, pH adjustment agents, conditioning agents, anti-cracking/hardening agents, anti-mushing/sloughing agents and active ingredients. Suitable plasticizers, fragrances, dyes, opacifiers, pearlizers, fillers, chelating agents, pH adjustment agents, conditioning agents, anti-cracking/hardening agents, anti-mushing/sloughing agents and active ingredients would be known to the person skilled in the art.

Such additional ingredients may be selected from one or more of polyalkylene glycols and derivatives; starches and dextrins, maltodextrin and other carbohydrates; inorganic particulate materials for example talc, kaolin, bentonite clay, aluminosilicate clays or other clays; carbonate or sulphate salts; glycerol esters or ethylene glycol esters; sugars and crystalline polyols; other waxes and fatty alcohols; germicides; antimicrobial agents; antioxidants; cationic polymers.

Suitable chelating agents include aminocarboxylic acid chelating agents and amino phosphonic acid chelating agents. Polycarboxylic acid derived acid chelating agents are also suitable for use herein.

Suitable aminocarboxylic acid chelating agents may be selected from one or more of ethylenediaminotetraacetic acid (EDTA), ethyenetriamine pentaacectic acid, ethylenediaminediglutaric acid, 2-hydroxypropylenediamine disuccinic acid, diethylene triamine pentaacetic acid (DTPA), N-hydroxyethylethylenediamine triacetic acid, ethylenediamine tetrapropionic acid, triethylenetetraaminehexa-acetic acid, ethanol-diglycines, propylene diamine tetracectic acid (PDTA), methyl glycine diacectic acid (MGDA), and Tetrasodium glutamate diacetate (GLDA).

Preferred chelating agents include sodium ethylenediaminetetraacetate (EDTA) and trisodium ethylenediamine disuccinate (EDDS).

Suitable opacifiers and pearlizers include titanium dioxide, zinc stearate, magnesium stearate, ethylene glycol monostearate (EGMS) or ethylene glycol distearate (EGDS).

Suitable anti-cracking/hardening agents include sodium lactate, hydrogenated vegetable oil, hydrogenated castor oil and polyethylene glycol.

Suitable anti-mushing/sloughing agents include aluminium triformate.

In embodiments wherein the solid cleansing composition is a personal cleansing composition, suitably a personal cleansing bar, suitable active ingredients of component (d) may be skin benefit agents and/or hair benefit agents.

Suitable skin and hair benefit agents include silicones (e.g. dimethicone), glycerol, propylene glycol, dipropylene glycol, urea, emollient esters (e.g. isopropyl myristate, petrolatum, heptyl undecylate, diheptyl succinate, neopentyl glycol diheptanoate, C₁₂₋₁₅ alkyl benzoate, neopentyl glycol dicaprate, oleyl oleate, cetearyl ethylhexanoate, ethylhexyl palmitate), tocopherol acetate, anti-bacterial agents (e.g. triclosan, triclocarban, chloroxylenol, benzalkonium chloride, benzethonium chloride), protein hydrolysates, allantoin, aloe vera, caffeine and exfoliating agents (e.g. microbeads, jojoba beads, polylactic acid beads, loofah fibres, ground nut shells).

Suitable hair benefits agents include anti-dandruff agents (e.g. zinc pyrithione, piroctone olamine, ketoconazole, salicylic acid, selenium sulfide), hair reactive bonding agents for hair repair, hair strengthening or dyed-hair colour protection (e.g. alpha-hydroxy aldehydes, alpha-hydroxy ketones, dialkyl maleates) and keratin hydrolysate.

In embodiments wherein the solid cleansing composition is a laundry cleansing composition, suitably a laundry cleansing bar, suitable active ingredients of component (d) will be known to the person skilled in the art, including chelating agents, builders, bleaching agents, bleach activators, pigments, dispersants, polymeric dispersing agents, redeposition additives, dyes, dye transfer inhibitors, fragrances, fragrance delivery systems, enzymes, enzyme stabilizers, biocides, probiotics, preservatives, pH adjusting agents, phosphates, silicates, zeolites, peroxide based compounds (especially hydrogen peroxide, chlorine bleaches), perborate compounds, percarbonate compounds, bleach activators and catalysts, cationic surfactants, redeposition additives, brighteners, sud suppressors, fabric softeners and hydrotropes.

In embodiments wherein the solid cleansing composition is a hard surface cleaning composition, suitably a hard surface cleaning bar, suitable active ingredients of component (d) will be known to the person skilled in the art, including builders, bleaches, bleach activators, redeposition additives, dye transfer inhibitors, enzymes, colorants and fragrances.

In some embodiments, component (d) does not comprise any polyalkylene oxides, for example no polyalkylene glycols. Suitably the solid cleansing composition of this first aspect suitably comprises less than 5 wt % polyalkylene oxides, suitably less than 3 wt % or less than 1 wt % polyalkylene oxides. Suitably the solid cleansing composition does not comprise polyalkylene oxides.

In some embodiments, the solid cleansing composition of this first aspect suitably comprises less than 5 wt % polyalkylene glycols, suitably less than 3 wt % or less than 1 wt % polyalkylene glycols. Suitably the solid cleansing composition does not comprise polyalkylene glycols.

In particular, the solid cleansing composition of this first aspect suitably comprises less than 5 wt % polyalkylene glycols which have a molecular weight in the range 1,500-10,000, suitably less than 3 wt % or less than 1 wt % polyalkylene glycols having said molecular weight. Suitably the solid cleansing composition does not comprise polyalkylene glycols which have a molecular weight in the range 1,500-10,000.

Suitably component (d) is present in an amount of at least 2 wt % of the solid cleansing composition, suitably at least 4 wt % or at least 5 wt %.

Suitably component (d) is present in an amount of up to 15 wt % of the solid cleansing composition, suitably up to 12 wt % or up to 10 wt %.

Suitably component (d) is present in an amount of from 1 to 15 wt % of the solid cleansing composition, suitably from 2 to 12 wt % or from 4 to 10 wt %.

Suitably components (a), (b), (c) and (d) together provide at least 85 wt % of the solid cleansing composition, suitably at least 90 wt % or at least 95 wt %.

The solid cleansing composition of this first aspect suitably comprises less than 10 wt % water, suitably less than 8 wt % or less than 5 wt % water. Preferably the solid cleansing composition comprises from 3 to 6 wt % water.

The solid cleansing composition of this first aspect suitably comprises less than 3 wt % salt, suitably less than 2 wt % or less than 1 wt % salt.

Suitably the solid cleansing composition contains no added water. Any water present in the solid cleansing composition is present in the components (a), (b), (c) or (d) and no additional water is added during manufacture of the solid cleansing composition.

Suitably the solid cleansing composition of this first aspect, as a whole, has a percentage renewable carbon index (% RCI) of at least 70%, at least 80%, at least 90% or at least 98%, preferably at least 99%. Preferably the solid cleansing composition has a % RCI of from 95 to 100%, preferably from 99 to 100%. Preferably the solid cleansing composition has a % RCI of approximately 100%.

The solid cleansing composition of this first aspect is suitably in the form of a bar. The bar suitably has a size and shape appropriate for use by a consumer as a personal cleansing bar, a laundry bar or a hard surface cleansing bar as is common in the industry. The solid cleansing composition may therefore be referred to as a personal cleansing bar, a laundry bar or a hard surface cleansing bar. Suitably the solid cleansing composition is a personal cleansing bar.

The second aspect of the present invention provides a method of forming a solid cleansing composition according to the first aspect, the method comprising the steps of:
(i) forming a first surfactant composition by reacting an excess of one or more fatty acids of formula R¹COOH, or a reactive equivalent thereof, with a compound of formula (II):

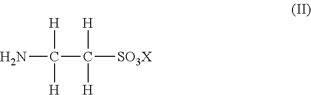

wherein X is hydrogen, a metal ion or an optionally substituted ammonium ion; and R¹ is an optionally substituted C₃-C₃₅ hydrocarbyl group;
wherein the first surfactant composition comprises:
   (a) at least 45 wt % of one or more taurate surfactants of formula (I):

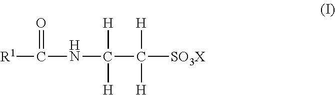

wherein X is hydrogen, a metal ion or an optionally substituted ammonium ion; and R¹ is an optionally substituted C₃-C₃₅ hydrocarbyl group; and
   (b) at least 20 wt % of the one or more fatty acids of formula R¹COOH;
and
(ii) combining the first surfactant composition formed in step (i) with at least one of:
   (c) at least one secondary surfactant, preferably selected from a non-ionic surfactant, a zwitterionic surfactant, a cationic surfactant or an anionic surfactant which is not a taurate surfactant of formula (I), or a mixture thereof; or
   (d) at least one additional ingredient selected from plasticizers, fragrances, dyes, opacifiers, pearlizers, fillers, chelating agents, pH adjustment agents, cationic polymers, conditioning agents, anti-cracking/hardening agents, anti-mushing/sloughing agents and active ingredients;
to form the solid cleansing composition.

Components (a), (b), (c) when present and (d) when present may have any of the suitable features and advantages discussed above in relation to the first aspect.

Suitably step (i) involves reacting the one or more fatty acids of formula R¹COOH or a reactive equivalent thereof, with the compound of formula (II) in a molar ratio of from 10:1 to 1:1, or from 10:1 to 1.1:1, suitably from 6:1 to 2:1. Therefore, step (i) suitably involves reacting an excess of the one or more fatty acids of formula R¹COOH or a reactive equivalent thereof, with the compound of formula (II), suitably at least two equivalents of the one or more fatty acids of formula R¹COOH with one equivalent of the compound of formula (II).

Suitably step (i) involves reacting the one or more fatty acids of formula R¹COOH or a reactive equivalent thereof, with the compound of formula (II) at elevated temperature, suitably at least 50° C. or at least 100° C. In some embodiments the temperature is at least 150° C. or at least 175° C. The reaction suitably involves the use of a catalyst, for example an acid. Suitable catalysts/acids are known to the skilled person.

The first surfactant composition formed in step (i) comprises component (a) in an amount of at least 45 wt %, suitably at least 50 wt % or at least 55 wt % of the first surfactant composition.

Suitably the first surfactant composition formed in step (i) comprises component (a) in an amount of up to 80 wt %, suitably up to 75 wt % or up to 70 wt % of the first surfactant composition.

Suitably the first surfactant composition formed in step (i) comprises component (a) in an amount of from 45 to 80 wt %, suitably from 50 to 75 wt % of the first surfactant composition, suitably from 55 to 70 wt % of the first surfactant composition.

The first surfactant composition formed in step (i) comprises component (b) in an amount of at least 20 wt %, suitably at least 25 wt % or at least 30 wt % of the first surfactant composition.

Suitably the first surfactant composition formed in step (i) comprises component (b) in an amount of up to 50 wt %, suitably up to 45 wt % or up to 40 wt % of the first surfactant composition.

Suitably the first surfactant composition formed in step (i) comprises component (b) in an amount of from 20 to 50 wt %, suitably from 25 to 45 wt %, suitably from 30 to 40 wt % of the first surfactant composition.

Suitably component (a) and (b) are present in the first surfactant composition in a weight ratio of from 4:1 to 1:2, suitably from 3:1 to 1:1, from 2:1 to 1:1 or from 2:1 to 1.5:1.

Suitably the first surfactant composition consists essentially or consists of components (a) and (b).

Suitably the first surfactant composition formed in step (i) is a solid at room temperature. In some embodiments, the first surfactant composition formed in step (i) is in solid form when combined with at least one of components (c) and (d) in step (ii) to form the solid cleansing composition.

In some embodiments, the first surfactant composition formed in step (i) is in liquid form when combined with at least one of components (c) and (d) in step (ii) to form the solid cleansing composition. In such embodiments, the first surfactant composition formed in step (i) may be combined with at least one of components (c) and (d) in step (ii) whilst the first surfactant composition is in a liquid or molten form before it solidifies. Alternatively, the first surfactant composition formed in step (i) may be a solid composition which is then be melted to form a liquid composition and then combined with at least one of components (c) and (d) in step (ii), followed solidification into the solid cleansing composition.

Suitably the first surfactant composition formed in step (i) is in the form of solid flakes.

In some embodiments, the solid cleansing composition produced by the method may comprise a component (c) at least one secondary surfactant, as described above in relation to the solid cleansing composition of the first aspect.

The at least one secondary surfactant may be selected from acyl isethionate, acyl alkyl isethionate and acyl alkyl taurate surfactants, or mixtures thereof, as described above in relation to the first aspect. Suitably, the at least one secondary surfactant is selected from sodium lauroyl isethionate, sodium cocoyl isethionate, sodium lauroyl methyl isethionate, sodium cocoyl methyl isethionate sodium lauroyl methyl taurate and sodium cocoyl methyl taurate, or mixtures thereof.

In such embodiments, step (i) may involve forming the at least one secondary surfactant (c), suitably in the same reaction used to provide components (a) and (b), to provide the first surfactant composition comprising components (a), (b) and (c). The reaction may be a “one-pot” reaction in which components (a), (b) and (c) are formed simultaneously from the respective reactants, component (b) being unreacted fatty acid. In such a reaction, the fatty acid is used in an excess compared to the combined amount of the compound of formula (II) and the compound used to form component (c).

In such embodiments, step (i) may involve reacting an excess of the one or more fatty acids of formula R¹COOH or a reactive equivalent thereof, with:
a compound of formula (II):

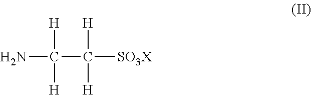

wherein X is hydrogen, a metal ion or an optionally substituted ammonium ion; and R¹ is an optionally substituted C₃-C₃₅ hydrocarbyl group;
and at least one of:
   a compound of formula (V):

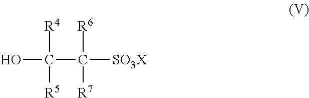

wherein each of R⁴, R⁵, R⁶ and R⁷ independently represents a hydrogen atom or a C₁-4 alkyl group; and X is as defined above for the X group of component (a) of formula (I);
and
a compound of formula (VI):

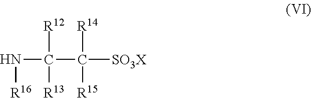

wherein X is as defined above in relation to X of component (a) of formula (I); each of R¹², R¹³, R¹⁴ and R¹⁵ independently represents hydrogen or an optionally substituted C₁-C₄ alkyl group;
and R¹⁶ is an optionally substituted C₁-C₄ alkyl group.

The X, R⁴, R⁵, R⁶, R⁷, R¹², R¹³, R¹⁴, R¹⁵ and R¹⁶ groups are suitably as defined in relation to compounds of formulas (III) and (IV) above. Preferably said groups are selected to produce component (c) selected from sodium lauroyl isethionate, sodium cocoyl isethionate, sodium lauroyl methyl isethionate, sodium cocoyl methyl isethionate sodium lauroyl methyl taurate and sodium cocoyl methyl taurate, or mixtures thereof.

Suitably said reaction produces the first surfactant composition comprising components (a), (b) and (c) as described above.

In such embodiments, step (i) suitably involves reacting the one or more fatty acids of formula R¹COOH or a reactive equivalent thereof, with the compound of formula (II) and either or both of the compounds of formulas (V) and (VI) at elevated temperature, suitably at least 50° C. or at least 100° C. The reaction suitably involves the use of a catalyst, for example an acid. Suitable catalysts/acids are known to the skilled person.

In step (i), the compound of formula (II) and the compounds of formulas (V) and (VI), when present, may be used in relative proportions sufficient to provide the amounts of components (a), (c1) and (c2) in the first surfactant composition as described below.

Suitably step (i) involves reacting the one or more fatty acids of formula R¹COOH or a reactive equivalent thereof, with the compounds of formula (II) and at least one of the compounds of formulas (V) and (VI), when considered together, in a molar ratio of from 10:1 to 1:1, or from 10:1 to 1.1:1, suitably from 6:1 to 2:1. Therefore, the method suitably involves reacting an excess of the one or more fatty acids of formula R¹COOH or a reactive equivalent thereof, with the compounds of formula (II), (V) and (VI), taken together, suitably at least two equivalents of the one or more fatty acids of formula R¹COOH with one equivalent of the compounds of formula (II), (V) and (VI), taken together.

Suitably in step (i) the molar ratio of the amount of the compound of formula (II) to the amount of compounds of formulas (V) and (VI), taken together, used in the reaction is from 20:1 to 2:1, suitably from 10:1 to 2:1 or from 5:1 to 3:1 or from 2.5:1 to 1:1.

Suitably in step (i) the molar ratio of the amount of compounds of formula (V) to the amount of compounds of formula (VI), when both are used in the reaction, is from 20:1 to 1:20, suitably from 10:1 to 1:10 or from 5:1 to 1:5.

Therefore step (i) suitably provides a first surfactant composition comprising:
(a) at least 45 wt % of one or more taurate surfactants of formula (I):

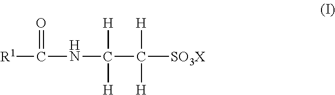

wherein X is hydrogen, a metal ion or an optionally substituted ammonium ion; and R¹ is an optionally substituted C₃-C₃₅ hydrocarbyl group; and
(b) at least 20 wt % of one or more fatty acids of formula R¹COOH;
(c) at least 1 wt % of a secondary surfactant selected from:
   (c1) an isethionate surfactant of formula (III):

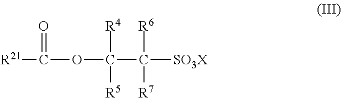

wherein R²¹ is as defined above for R¹ of component (a) of formula (I); each of R⁴, R⁵, R⁶ and R⁷ independently represents a hydrogen atom or a C₁-4 alkyl group; and X is as defined above for the X group of component (a) of formula (I); and
   (c2) an acyl alkyl taurate surfactant of formula (IV):

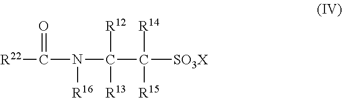

wherein X and R²² are suitably as defined above in relation to R¹ and X of component (a) of formula (I); wherein R¹², R¹³, R¹⁴ and R¹⁵ are each independently selected from hydrogen or an optionally substituted C₁-C₄ alkyl group; and R¹⁶ is an optionally substituted C₁-C₄ alkyl group;
or a mixture thereof.

In some embodiments, R²¹ of the compound of formula (III) is the same as R¹ of component (a) of formula (I) and/or R²² of the compound of formula (IV) is the same as R¹ of component (a) of formula (I).

Suitably component (c) is present in an amount of at least 2 wt % of the first surfactant composition, suitably at least 3 wt %.

Suitably component (c) is present in an amount of up to 10 wt % of the first surfactant composition, suitably up to 8 wt % or up to 6 wt %.

Suitably component (c) is present in an amount of from 1 to 10 wt % of the first surfactant composition, suitably from 2 to 8 wt % or from 3 to 6 wt %.

Suitably components (a), (b), (c1) and/or (c2) together provide at least 85 wt % of the first surfactant composition, suitably at least 90 wt % or at least 95 wt %.

Suitably such first surfactant compositions formed in step (i) are in the form of solid flakes.

In some embodiments, the first surfactant composition formed in step (i) comprises both secondary surfactants (c1) and (c2). In such embodiments, the amounts of component (c) present in the first surfactant composition referred to above relate to the total amount of secondary surfactants (c1) and (c2) present.

In alternative embodiments, step (i) may involve blending the at least one secondary surfactant (c), for example the secondary surfactants (c1) and/or (c2) discussed above, with components (a) and (b) to provide the first surfactant composition comprising component (c).

Step (ii) of the method of this second aspect involves combining the first surfactant composition formed in step (i) with at least one of components (c) and (d).

In some embodiments, step (ii) may involve combining the first surfactant composition formed in step (i) with at least one of components (c) and (d), in the solid phase. For example, step (ii) may involve combining the first surfactant composition with components (c) and/or (d) as solids in an extruder. This step may be part of a conventional soap bar making process as discussed further below.

In some embodiments, step (ii) may involve melting the first surfactant composition comprising components (a) and (b) to provide a molten first surfactant composition and then combining said molten first surfactant composition with components (c) and/or (d) to provide a molten cleansing composition. This step may be part of a so-called “melt and pour” process for forming a solid cleansing composition, such as a personal cleansing bar, as discussed further below.

Suitably the method of this second aspect comprises the step of:
(iii) forming the solid cleansing composition into a bar.

Step (iii) suitably involves conventional soap bar making processes to form the solid cleansing composition obtained in step (ii) of the method of this second aspect into a bar, suitably a personal cleansing bar. For example, step (iii) may involve extruding (or plodding) the solid cleansing composition and cutting (or stamping) to the required size and shape of bar.

Alternatively, step (iii) may involve melt casting the solid cleansing composition to form the bar of the required size and shape. In such embodiments, step (ii) involves forming a molten cleansing composition as described above and step (iii) involves pouring said molten cleansing composition into a mould of the required size and shape of bar and then allowing the molten cleansing composition to solidify to form the solid cleansing composition as a bar. Such a process may be referred to as a “melt and pour” process for forming a solid cleansing composition in bar form.

Alternatively, step (iii) may involve compacting the solid cleansing composition under pressure to form the bar of the required size and shape, for example in a suitable mould. In such embodiments, step (ii) may involve combining the first surfactant composition formed in step (i), for example in powder, pellet, noodle or flake form, with at least one of components (c) and (d), suitably in the solid phase. Said combination of the solid surfactant and the at least of components (c) and (d) is then compacted in step (iii) to form the bar.

The third aspect of the present invention provides a method of cleansing skin and/or hair, the method comprising contacting the skin and/or hair with a solid cleansing composition according to the first aspect.

Suitably the solid cleansing composition is in bar form. Suitably the method involves contacting the solid cleansing composition with water and said skin and/or hair.

Suitably the solid cleansing composition is formed by a method according to the second aspect of the present invention.

The fourth aspect of the present invention provides a use of a solid cleansing composition according to the first aspect for cleansing skin and/or hair.

Suitably the solid cleansing composition is in bar form. Suitably the use involves contacting the solid cleansing composition with water and said skin and/or hair.

Suitably the solid cleansing composition is formed by a method according to the second aspect of the present invention.

The fifth aspect of the present invention provides a cleansing product comprising a solid cleansing composition according to the first aspect and a packaging.

Suitably the cleansing product is a personal cleansing product, suitably intended for use to clean skin and/or hair of a consumer.

Suitably the solid cleansing composition is formed by a method according to the second aspect of the present invention.

Suitably the solid cleansing composition is in bar form. Suitably the packaging encloses an individual bar. The packaging may be a box, bag or wrapper. The packaging may be made of any suitable material, for example cardboard, paper, metallic foil, a solid plastics material or plastic film. Preferably the packaging is made from a recycled, recyclable and/or biodegradeable material. Suitably the packaging is formed from a renewable material, suitably paper or cardboard.

The sixth aspect of the present invention provides a method of cleansing fabrics, suitably soiled fabrics or hard surfaces, the method comprising contacting the fabric or hard surface with a solid cleansing composition according to the first aspect. Suitably the solid cleansing composition is a laundry cleansing composition or a hard surface cleaning composition as described in relation to the first aspect.

The seventh aspect of the present invention provides a use of a solid cleansing composition according to the first aspect for cleansing fabrics, suitably soiled fabrics or hard surfaces. Suitably the solid cleansing composition is a laundry cleansing composition or a hard surface cleaning composition as described in relation to the first aspect.

### EXAMPLES

### Example 1

The solid cleansing composition of Example 1, in the form of a cleansing bar, was prepared as follows.

A sodium cocoyl taurate reaction product (comprising approximately 10 wt % unreacted coconut fatty acid) was combined with further (additional) coconut fatty acid to provide a mixture containing 65 wt % of sodium cocoyl taurate and 35 wt % of free fatty acid (coconut fatty acids). This mixture was heated to approximately 210° C. and then poured onto a stainless steel flaking sheet to harden. The hardened mixture was then flaked using standard techniques to provide a flaked solid cleansing composition comprising approximately 65 wt % of sodium cocoyl taurate and 35 wt % of free fatty acid (coconut fatty acids).

The flaked solid cleansing composition was formulated with the other components listed in Table 1 below and processed into the solid cleansing bar of Example 1, using the following procedure. Table 1 shows the relative amounts of the components used.

TABLE 1

| Component | Amount (wt %) |
|---|---|
| flaked solid cleansing composition | 95.80 |
| (SCT/coconut fatty acid) | |
| TiO₂ powder | 0.20 |
| Water | 3.00 |
| Fragrance | 1.00 |

SCT = sodium cocoyl taurate

Mixing procedure:
1. Add flaked solid cleansing composition to an amalgamator or a hand mixing vessel.
2. Disperse TiO2 evenly over the flake/noodles. Mix until uniformly distributed.
3. Add liquids (water and fragrance), mix until liquid has coated the flakes (30 sec to 1 min)
4. Material is ready to be refined.

Refining procedure:
1. Add material to an extruder/plodder with only the noodle plate attached.
2. Begin plodding with no heat or cooling on the barrel.
3. Once material starts to flow out of the noodle plate turn on cooling jacket to cool material at 5-15° C.
4. After the first pass through the noodle plate, take the material and pass through noodle plate again.
5. Add a 10 or 20 mesh screen.
6. Pass material through screen twice.
7. Remove screen and fit a 20 or 30 mesh screen.
8. Pass material through screen twice.
9. Attach extrusion head, heat extrusion head to 60° C.
10. Extruding until the billets are solid and smooth.
11. Press the bars with soap die.

### Comparative Example 1

A comparative example solid cleansing bar (Comparative Example 1) was formed using a similar procedure using sodium cocoyl methyl taurate to replace the sodium cocoyl taurate.

The compositions of Example 1 and Comparative Example 1 (Comp. Ex. 1) are shown in Table 2 below.

TABLE 2

| | Example 1 | Comp. Ex. 1 |
|---|---|---|
| Component | (wt % active) | (wt % active) |
| Sodium cocoyl taurate | 62.3 | |
| Sodium cocoyl methyl | | 62.3 |
| taurate | | |
| Coco fatty acid | 33.5 | 33.5 |
| Water | 3 | 3 |
| Fragrance | 1 | 1 |
| Titanium dioxide | 0.2 | 0.2 |

### Hardness Tests

The hardness of the solid cleansing bars of Example 1 and Comparative Example 1 was tested using the following method (according to ASTM D1321-10 Standard Test Method for Needle Penetration of Petroleum Waxes).

### Equipment:

Penetrometer—Precision Scientific Instrument Company, Cat. No. 73510 or equivalent.

Timer—A stop watch capable of accurately displaying a 5-second interval.

Needle and Plunger—Standard Needle K 17700 supplied by Koehler Instruments as described in ASTM D1321. Plunger is as that described in the same standard. Supplied 50-gram mass plus standard needle and plunger shall equal 100 grams total mass.

### Method:

1. Bar soap samples allowed to equilibrate in the testing room at 24+/−2° C. for at least 1 hour.
2. Place the bar specimen on the penetrometer base. Make certain the sample cannot teeter during testing.
3. Make certain the 50 g mass is placed above the needle and plunger assembly making a total load of 100+/−0.15 g.
4. Adjust the shaft so that the indictor on the scale set in the ‘Zero’ position.
5. Using the knurled knob on the side of the penetrometer, adjust the tip of the needle so it nearly touches the surface of the specimen.
6. Watch the ‘reflection’ of the needle tip (use a light to cast a long shadow of the needle tip) as needle is slowly adjusted to nearly touch the surface of the test bar.
7. Release the needle shaft using thumb and forefinger.
8. Squeeze and hold the levers for 5.0+/−0.1 seconds while timing this interval with an accurate digital stop watch, releasing the levers after the 5 second interval.
9. Gently depress the indicator shaft, until it stops, and read the penetration from the indicator scale.
10. Make four tests at points equally spaced (if possible) across the surface of the sample.
11. In many cases, a flat bar sample may be tested on the diagonal, for 3 readings, and then in a separate corner for the final reading (four readings total).
12. Record the average scale readings for the four penetrations and report to the nearest penetrometer unit (e.g. Average=40.4 reported as 40).

### Calculation:

Report average of 4 readings and report results to the nearest penetrometer unit.

### Foaming Tests

The foaming performance of the solid cleansing bars of Example 1 and Comparative Example 1 was tested using the following method.
1. Bar pretreatment: Wearing disposable gloves, worn inside out and well-washed in plain soap, wash down all test bars for at least 1 minute before starting the test sequence. This is best done by twisting the bars about 20 times under running water.
2. Place about 5 liters of water of known hardness and at a specified temperature (typically 20-40° C.) in a bowl. Change the water after each cleansing bar has been tested.
3. Take up the bar, dip it in the water, and remove it. Rotate the bar 20 times between the hands. Replace the bar on a soap dish.
4. The lather is generated from the material remaining on the gloves:
   Stage 1: Rub the tips of the fingers of one hand (either hand) on the palm of the other hand 10 times.
   Stage 2: Grip the right hand with the left, or vice versa, and force the lather to the tips of the fingers. Repeat with the hands reversed. This operation is repeated five times with each hand.
5. Repeat stages 1 and 2.
6. Place the lather in a calibrated beaker.
7. Repeat the whole procedure of lather generation twice more, combining all the lather in the beaker.
8. Stir the combined lather gently to release large pockets of air. Read and record the volume.

### Rate of Wear Tests

The rate of wear of the solid cleansing bars of Example 1 and Comparative Example 1 was tested using the following method.

Wet a pre-weighed bar in running water and rotate 20 times while in the tester's hands. Place on a support stand. Repeat 10 times at half-hour intervals. Weigh the washed bar after allowing it to dry at room temperature for 16 hours. The weight change multiplied by 100 and divided by the initial weight of the bar denotes the percent rate of wear of the bar.

### Results

The results of the hardness, foaming and rate of wear tests are shown below in Table 3 for the solid cleansing bars of Example 1 and Comparative Example 1.

TABLE 3

| | Example 1 | Comp. Ex. 1 |
|---|---|---|
| Hardness | 8 | 15 |
| Foaming (ml) | 250 | 175 |
| Rate of wear (%) | 16.70 | 14.70 |

These results show that the solid cleansing bar of Example 1 was harder, higher foaming and had a higher wear rate than the solid cleansing bar of Comparative Example 1. Higher foaming and faster wearing may be beneficial in some applications. High foaming is considered to be a desirable property of personal cleansing bars in particular. Faster wearing, within reasonable limits, may provide more surfactant per use and therefore may provide a more effective cleansing performance. The present invention may therefore provide a potentially advantageous solid cleansing composition, whilst improving the sustainability of the composition due to the high % RCI of the acyl taurate surfactants used in the composition.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

For the avoidance of doubt, wherein amounts of components in a composition are described in wt %, this means the weight percentage of the specified component in relation to the whole composition referred to. For example, “component (a) is present in an amount of from 40 to 80 wt % of the solid cleansing composition” means that 40 to 80 wt % of the solid cleansing composition is provided by component (a).

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A solid cleansing composition comprising:
(a) at least 35 wt % of one or more taurate surfactants of formula (I): 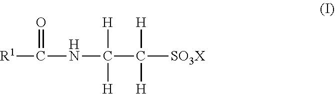
wherein X is hydrogen, a metal ion or an optionally substituted ammonium ion; and R¹ is an optionally substituted C₃-C₃₅ hydrocarbyl group; and
(b) at least 15 wt % of one or more fatty acids.

2. The solid cleansing composition according to claim 1, wherein R¹ is an optionally substituted C₇-C₁₇ hydrocarbyl group; preferably a C₇-C₁₇ alkyl group.

3. The solid cleansing composition according to claim 1, wherein R¹ is provided by one or more naturally occurring fatty acids.

4. The solid cleansing composition according to claim 1, wherein R¹ is provided by lauric acid or is provided by fatty acids obtained from coconut oil and/or palm kernel oil.

5. The solid cleansing composition according to claim 1, wherein the fatty acid of component (b) has the formula R²COOH, wherein R² is as defined in relation to R¹ of component (a) of formula (I).

6. The solid cleansing composition according to claim 5, wherein R² of R²COOH is the same as R¹ of component (a) of formula (I).

7. The solid cleansing composition according to claim 1, wherein component (a) is sodium cocoyl taurate or sodium lauroyl taurate.

8. The solid cleansing composition according to claim 1, wherein component (b) is coconut fatty acid or lauric acid.

9. The solid cleansing composition according to claim 1, wherein component (a) and (b) together provide at least 80 wt % of the solid cleansing composition.

10. The solid cleansing composition according to claim 1, wherein component (a) and (b) are present in the solid cleansing composition in a weight ratio of from 3:1 to 1:1.

11. The solid cleansing composition according to claim 1, comprising:
(c) at least 1 wt % of at least one secondary surfactant, preferably selected from a non-ionic surfactant, a zwitterionic surfactant, a cationic surfactant or an anionic surfactant which is not a taurate surfactant of formula (I), or a mixture thereof.

12. The solid cleansing composition according to claim 11, wherein the secondary surfactant is selected from:
(c1) an isethionate surfactant of formula (III): 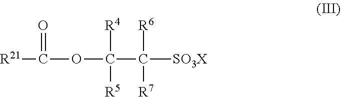
wherein R²¹ is an optionally substituted C₃-C₃₅ hydrocarbyl group; each of R⁴, R⁵, R⁶ and R⁷ independently represents a hydrogen atom or a C₁-4 alkyl group; and X is hydrogen, a metal ion or an optionally substituted ammonium ion; and
(c2) an acyl alkyl taurate surfactant of formula (IV): 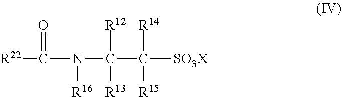
wherein R²² is an optionally substituted C₃-C₃₅ hydrocarbyl group; wherein R¹², R¹³, R¹⁴ and R¹⁵ are each independently selected from hydrogen or an optionally substituted C₁-C₄ alkyl group; and R¹⁶ is an optionally substituted C₁-C₄ alkyl group; and X is hydrogen, a metal ion or an optionally substituted ammonium ion;
or a mixture thereof.

13. The solid cleansing composition according to claim 1, comprising:
(d) at least 1 wt % of at least one additional ingredient selected from plasticizers, fragrances, dyes, opacifiers, pearlizers, fillers, chelating agents, pH adjustment agents, conditioning agents, anti-cracking/hardening agents, anti-mushing/sloughing agents and active ingredients.

14. The solid cleansing composition according to claim 1, comprising less than 10 wt % of fatty acid soap.

15. The solid cleansing composition according to claim 1, comprising less than 8 wt % water.

16. The solid cleansing composition according to claim 1, wherein component (a) has a percentage renewable carbon index (% RCI) of from 95 to 100%, preferably from 99 to 100%.

17. The solid cleansing composition according to claim 1, wherein the composition is in the form of a bar.

18. The solid cleansing composition according to claim 1, wherein components (a) and (b) are provided by reaction of an excess of one or more fatty acids of formula R¹COOH or a reactive equivalent thereof, with a compound of formula (II): 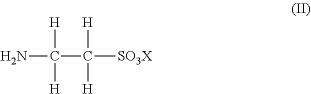
wherein X is hydrogen, a metal ion or an optionally substituted ammonium ion; and R¹ is an optionally substituted C₃-C₃₅ hydrocarbyl group.

19. A method of forming a solid cleansing composition according to claim 1, the method comprising the steps of:
(i) forming a first surfactant composition by reacting an excess of one or more fatty acids of formula R¹COOH, or a reactive equivalent thereof, with a compound of formula (II): 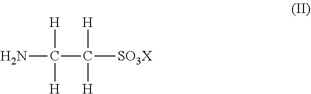
wherein X is hydrogen, a metal ion or an optionally substituted ammonium ion; and R¹ is an optionally substituted C₃-C₃₅ hydrocarbyl group;
wherein the first surfactant composition comprises:
(a) at least 45 wt % of one or more taurate surfactants of formula (I): 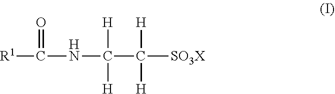
wherein X is hydrogen, a metal ion or an optionally substituted ammonium ion; and R¹ is an optionally substituted C₃-C₃₅ hydrocarbyl group; and
(b) at least 20 wt % of the one or more fatty acids of formula R¹COOH; and
(ii) combining the first surfactant composition formed in step (i) with at least one of:
(c) at least one secondary surfactant, preferably selected from a non-ionic surfactant, a zwitterionic surfactant, a cationic surfactant or an anionic surfactant which is not a taurate surfactant of formula (I), or a mixture thereof; or
(d) at least one additional ingredient selected from plasticizers, fragrances, dyes, opacifiers, pearlizers, fillers, chelating agents, pH adjustment agents, cationic polymers, conditioning agents, anti-cracking/hardening agents, anti-mushing/sloughing agents and active ingredients;
to form the solid cleansing composition.

20. The method according to claim 19, wherein the first surfactant composition formed in step (i) is in the form of solid flakes.

21. The method according to claim 19, comprising the step of:
(iii) forming the solid cleansing composition into a bar.

22. The method according to claim 19, wherein the solid cleansing composition comprises (c) at least 1 wt % of a secondary surfactant selected from:
(c1) an isethionate surfactant of formula (III): 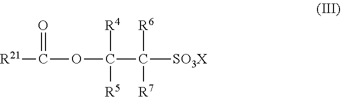
wherein X is hydrogen, a metal ion or an optionally substituted ammonium ion; R²¹ is an optionally substituted C₃-C₃₅ hydrocarbyl group; each of R⁴, R⁵, R⁶ and R⁷ independently represents a hydrogen atom or a C₁₋₄ alkyl group; or
(c2) an acyl alkyl taurate surfactant of formula (IV): 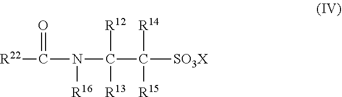
wherein X is hydrogen, a metal ion or an optionally substituted ammonium ion; and R²² is an optionally substituted C₃-C₃₅ hydrocarbyl group; wherein R¹², R¹³, R¹⁴ and R¹⁵ are each independently selected from hydrogen or an optionally substituted C₁-C₄ alkyl group; and R¹⁶ is an optionally substituted C₁-C₄ alkyl group;
or a mixture thereof.

23. The method according to claim 22, wherein step (i) involves forming the first surfactant composition by reacting the one or more fatty acids of formula R¹COOH, or a reactive equivalent thereof, with the compound of formula (II) and at least one of:
a compound of formula (V): 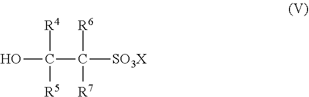
wherein X is hydrogen, a metal ion or an optionally substituted ammonium ion; and
wherein each of R⁴, R⁵, R⁶ and R⁷ independently represents a hydrogen atom or a C₁₋₄ alkyl group; or
a compound of formula (VI): 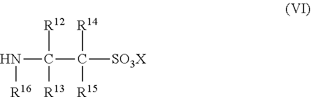
wherein X is hydrogen, a metal ion or an optionally substituted ammonium ion; each of R¹², R¹³, R¹⁴ and R¹⁵ independently represents hydrogen or an optionally substituted C₁-C₄ alkyl group; and R¹⁶ is an optionally substituted C₁-C₄ alkyl group;
or a mixture thereof.

24. A method of cleansing skin and/or hair, the method comprising contacting the skin and/or hair with a solid cleansing composition according to claim 1.

25. Use of a solid cleansing composition according to claim 1 for cleansing skin and/or hair.

26. A method of cleansing fabrics or hard surfaces, the method comprising contacting the fabric or hard surface with a solid cleansing composition according to claim 1.

27. Use of a solid cleansing composition according to claim 1 for cleansing fabrics or hard surfaces.

28. A cleansing product comprising a solid cleansing composition according to claim 1 and a packaging.
